(19) United States
(12) Patent Application Publication
(10) Pub. No.: US 2022/0079950 A1
(43) Pub. Date: Mar. 17, 2022

(54) THERAPEUTIC COMBINATIONS AS ANTIDOTES FOR ORGANOPHOSPHATE EXPOSURE

(71) Applicants: The Regents of the University of California, Oakland, CA (US); The Scripps Research Institute, La Jolla, CA (US)

(72) Inventors: Palmer TAYLOR, La Jolla, CA (US); Jeremiah MOMPER, La Jolla, CA (US); Zoran RADIC, La Jolla, CA (US); K. Barry SHARPLESS, La Jolla, CA (US); Rakesh SIT, La Jolla, CA (US)

(21) Appl. No.: 17/419,904

(22) PCT Filed: Dec. 31, 2019
(86) PCT No.: PCT/US2019/069088

§ 371 Date: Jun. 30, 2021

Related U.S. Application Data
(60) Provisional application No. 62/786,639, filed on Dec. 31, 2018.

(51) Int. Cl. A61K 31/55; A61K 45/06

(52) U.S. Cl. CPC A61K 31/55; A61K 45/06

(57) ABSTRACT

In alternative embodiments, the provided are therapeutic combinations comprising: nucleophilic hydroxyimino-acetamido alkylamine antidotes that cross the blood-brain barrier (BBB) to catalyze the hydrolysis of organophosphate (OP)-inhibited human acetylcholinesterase (hAChE) in the central nervous system (CNS); and, a brain efflux transporter inhibitor, or an inhibitor of renal tubular secretion, wherein optionally the brain efflux transporter inhibitor or the inhibitor of renal tubular secretion comprises a P-glycoprotein inhibitor, an organic anion transporter (OAT) inhibitor and/or an organic cation (OCT) transporter inhibitor.

### RELATED APPLICATIONS

This Patent Convention Treaty (PCT) International Application claims the benefit of priority to U.S. Provisional Application No. 62/786,639, filed Dec. 31, 2019. The aforementioned application is expressly incorporated herein by reference in its entirety and for all purposes.

### STATEMENT AS TO RIGHTS TO INVENTIONS MADE UNDER FEDERALLY SPONSORED RESEARCH

This invention was made with government support under NS058046 awarded by the National Institutes of Health. The government has certain rights in the invention.

### TECHNICAL FIELD

This invention relates to antidote chemistry for organophosphates and their detoxification. In alternative embodiments, the provided are therapeutic combinations comprising: nucleophilic hydroxyimino-acetamido alkylamine antidotes that cross the blood-brain barrier (BBB) to catalyze the hydrolysis of organophosphate (OP)-inhibited human acetylcholinesterase (hAChE) in the central nervous system (CNS); and, a brain efflux transporter inhibitor, or an inhibitor of renal tubular secretion, wherein optionally the brain efflux transporter inhibitor or the inhibitor of renal tubular secretion comprises a P-glycoprotein inhibitor, an organic anion transporter (OAT) inhibitor and/or an organic cation (OCT) transporter inhibitor.

### BACKGROUND

A recent spur of interest in centrally acting reactivators of organophosphate (OP)³ inhibited acetylcholinesterase (AChE) reflects a compelling need for antidotal therapy capable of efficient reinstatement of CNS AChE activity in OP-intoxicated individuals. Exposure to uncharged, lipophilic OPs from both pesticide and nerve agents leads to inhibition of peripheral and CNS AChE within minutes of exposure due to rapid OP distribution through the various body compartments of exposed individuals. There is always a need for functional improvement of antidotal therapies to enhance the concentrations retained in the central nervous system, particularly the brain.

### SUMMARY

In alternative embodiments, provided are therapeutic combinations comprising:

(a) (i) a compound as described in U.S. patent application publication no. US2015/0361060 A1 or WO/2014/127315 A1;

(ii) a compound having one of the following structures or compositions having one or more compounds of the following structures, or equivalents thereof, or a stereoisomer thereof, or an analog thereof, or a pharmaceutically acceptable salt thereof, or a bioisostere thereof: or,

(ii) a composition comprising an isolated compound consisting essentially of, or consisting of:

(1) a compound having the formula

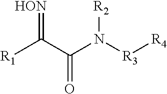

wherein:
R₁ is selected from the group consisting of: —H, -alkyl, and -aryl;
R₂ is selected from the group consisting of: —H, -alkyl, and -aryl;
R₃ is selected from the group consisting of: —(CH₂)ₙ—, and (CH₂)ₙ—CH(CH₃)—, wherein n is the integer 0, 1, 2, 3, 4 or 5;
R₄ is selected from the group consisting of: —H, -alkyl (wherein optionally the alkyl is selected from the group consisting of: -methyl, -ethyl, -propyl, -butyl, -i-propyl, and -i-butyl), -cycloalkyl (wherein optionally the cycloalkyl is selected from the group consisting of: -cyclopropyl, -cyclobutyl, -cyclopentyl, -cyclohexyl, -cycloheptyl, and -cyclooctyl), -aryl (wherein optionally the aryl is selected from the group consisting of: -phenyl, -naphthyl, -thienyl, and -indolyl), -saturated and nonsaturated heterocyclics (wherein optionally the saturated or nonsaturated heterocyclic is selected from the group consisting of: -aziridine, -oxirane-thiirane, -azirine, -oxirene, -thiirene, -azetidine, -oxetane, -thietane, -azete, -oxete, -thiete, -pyrrolidine, -oxolane, -thiolane, -pyrrole, -furan, -thiophene, -piperidine, -oxane, -thiane, -pyridine, -pyran, -thiopyran, -azepane, -oxepane, -thiepane, -azepine, -oxepine, -thiepine, -azocane, and -azocine), and bridged compounds (wherein optionally the bridged compound is selected from the group consisting of: -adamantanes, -amantadines, -biperidenes, -memantines, -methenamines, -rimantadines, -norbornanes, and -triazoles),

and optionally the terminal alkyl group R₄ is attached to an aminocarbonyl-aldoxime or a ketoaldoxime to form a bis-functional nucleophile, thereby statistically improving nucleophilic potential;

(2) a compound having the formula:

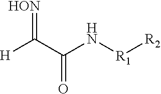

wherein
R₁ is selected from the group consisting of: —(CH₂)ₙ—, and (CH₂)ₙ—CH(CH₃)—, wherein n is the integer 0, 1, 2, 3, 4 or 5;
R₂ is selected from the group consisting of: —H, -alkyl (wherein optionally the alkyl is selected from the group consisting of: -methyl, -ethyl, -propyl, -butyl, -i-propyl, and -i-butyl), -cycloalkyl (wherein optionally the cycloalkyl is selected from the group consisting of: -cyclopropyl, -cyclobutyl, -cyclopentyl, -cyclohexyl, -cycloheptyl, and -cyclooctyl), -aryl (wherein optionally the aryl is selected from the group consisting of: -phenyl, -naphthyl, -thienyl, and -indolyl), -saturated and nonsaturated heterocyclics (wherein optionally the saturated or nonsaturated heterocyclic is selected from the group consisting of: -aziridine, -oxirane-thiirane, -azirine, -oxirene, -thiirene, -azetidine, -oxetane, -thietane, -azete, -oxete, -thiete, -pyrrolidine, -oxolane, -thiolane, -pyrrole, -furan, -thiophene, -piperidine, -oxane, -thiane, -pyridine, -pyran, -thiopyran, -azepane, -oxepane, -thiepane, -azepine, -oxepine, -thiepine, -azocane, and -azocine), and bridged compounds (wherein optionally the bridged compound is selected from the group consisting of: -adamantanes, -amantadines, -biperidenes, -memantines, -methenamines, -rimantadines, -norbornanes, and -triazoles);

(3) a compound having the formula:

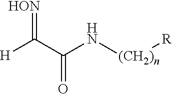

wherein
N is the integer 0, 1, 2, 3, 4, 5 or 6;
R is selected from the group consisting of: —H, -alkyl (wherein optionally the alkyl is selected from the group consisting of: -methyl, -ethyl, -propyl, -butyl, -i-propyl, and -i-butyl), -cycloalkyl (wherein optionally the cycloalkyl is selected from the group consisting of: -cyclopropyl, -cyclobutyl, -cyclopentyl, -cyclohexyl, -cycloheptyl, and -cyclooctyl), -aryl (wherein optionally the aryl is selected from the group consisting of: -phenyl, -naphthyl, -thienyl, and -indolyl), -saturated and nonsaturated heterocyclics (wherein optionally the saturated or nonsaturated heterocyclic is selected from the group consisting of: -aziridine, -oxirane-thiirane, -azirine, -oxirene, -thiirene, -azetidine, -oxetane, -thietane, -azete, -oxete, -thiete, -pyrrolidine, -oxolane, -thiolane, -pyrrole, -furan, -thiophene, -piperidine, -oxane, -thiane, -pyridine, -pyran, -thiopyran, -azepane, -oxepane, -thiepane, -azepine, -oxepine, -thiepine, -azocane, and -azocine), and bridged compounds (wherein optionally the bridged compound is selected from the group consisting of: -adamantanes, -amantadines, -biperidenes, -memantines, -methenamines, -rimantadines, -norbornanes, and -triazoles), and optionally the terminal alkyl group R is attached to an aminocarbonyl-aldoxime or a ketoaldoxime to form a bis-functional nucleophile, thereby statistically improving nucleophilic potential;

(4) a compound having the formula:

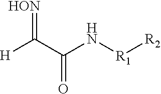

wherein
R₁ is selected from the group consisting of: —(CH₂)ₙ—, and (CH₂)ₙ—CH(CH₃)—,
wherein N is the integer 0, 1, 2, 3, 4 or 5;
R₂ is selected from the group consisting of:

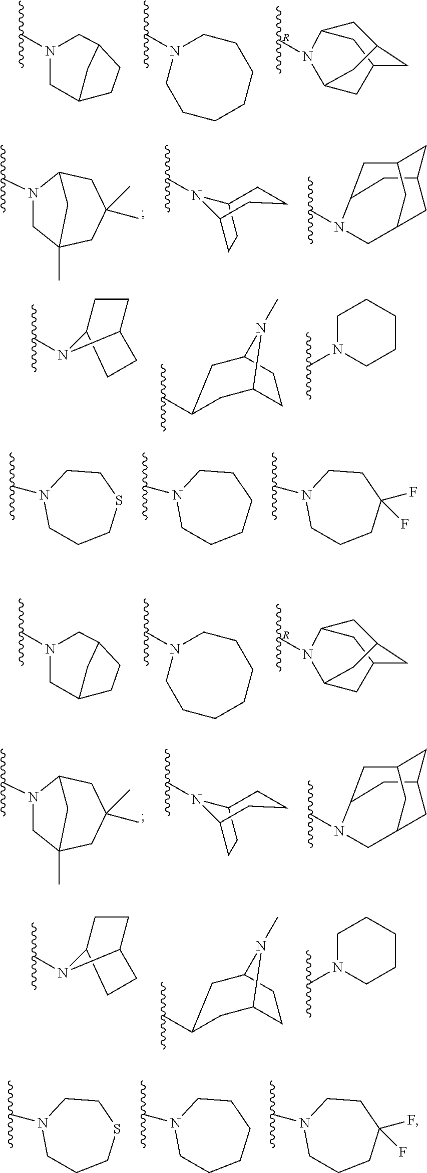

and any combination thereof;

(5) a compound having the formula:

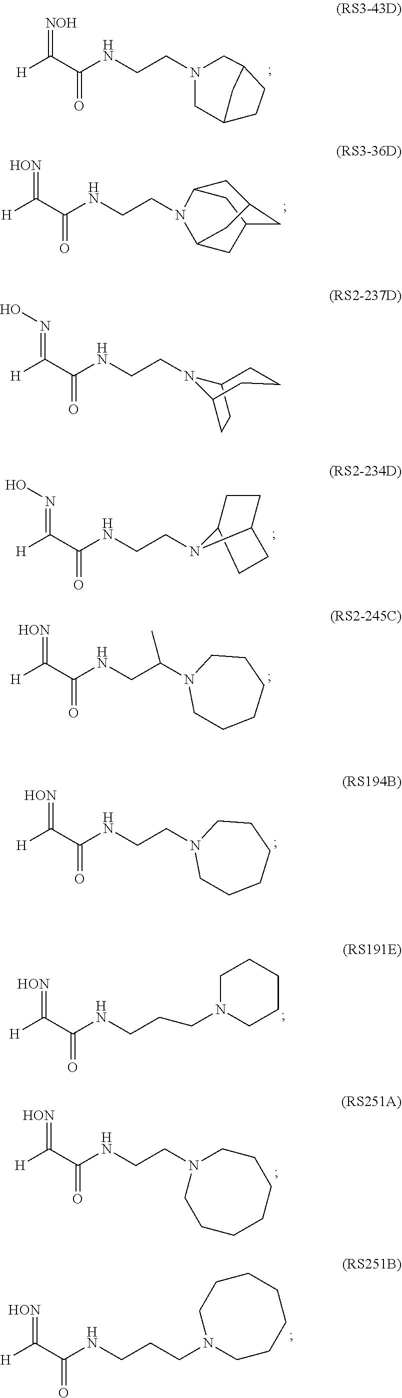

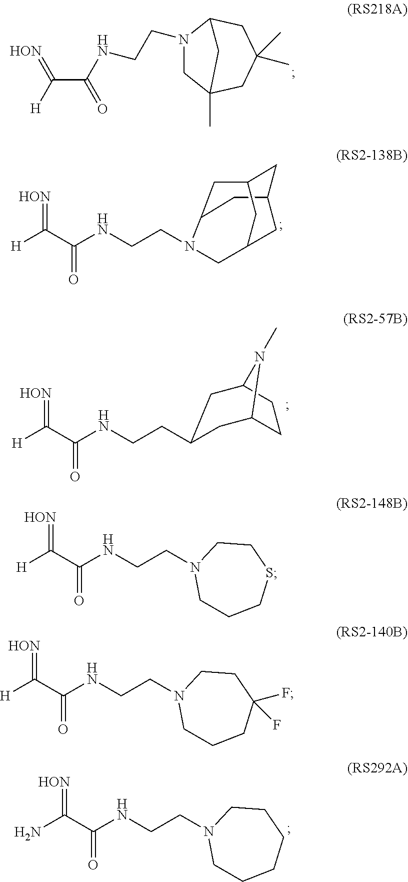

and

a combination thereof;

(6) compound having a structure as set forth in Table 1 (FIG. 3) to Table 2 (FIG. 4), or equivalents thereof, or pharmaceutically acceptable salt thereof,

wherein optionally the compound is RS2 138B, RS194B or RS191E;

wherein optionally the compound is RS251A, RS251B, RS218A, or RS2-57B; or

wherein optionally the compound is RS2-148B, RS2-140B, RS3-43D, RS3-36D, RS2-237D, RS2-234D or RS2-245C (FIG. 4A);

(7) any combination of any of the compounds of (1) to (6); or

(8) an isomer, an optical isomer or a stereoisomer; a racemate or a racemic mixture, an enantiomer, an individual diastereomer or a diastereomeric mixture or an analog, a crystalline product or a crystalline intermediate, a pharmaceutically acceptable salt, or a prodrug of the structure or compound, or a bioisostere of any compound or structure of (1) to (7); and

(b) a brain efflux transporter inhibitor, or an inhibitor of renal tubular secretion,

wherein optionally the brain efflux transporter inhibitor or the inhibitor of renal tubular secretion comprises or is an organic anion transporter (OAT) inhibitor and/or an organic cation (OCT) transporter inhibitor,

and optionally the brain efflux transporter inhibitor or the inhibitor of renal tubular secretion comprises or is:

(i) metformin (or GLUCOPHAGE™); a fluoroquinolone antibiotic (wherein optionally the fluoroquinolone is ciprofloxacin, or CILOXAN™, CIPRO™, or NEOFLOXIN™), norfloxacin (or NOROXIN™), levofloxacin (or LEVAQUIN™), or a combination thereof,

(ii) a p-glycoprotein inhibitor, wherein optionally the p-glycoprotein inhibitor is or comprises: tariquidar, clarithromycin (or BIAXIN™), erythromycin (or ERYTHROCIN™), ritonavir (or NORVIR™), verapamil (or CALAN™, COVERA™ VERELAN™, TARKA™), gallopamil (or methoxyverapamil), dimeditiapramine (or tiapamil), diltiazem (or CARDIZEM™), amiodarone (or CORDARONE™ NEXTERONE™), ciclosporin (or NEORAL™, SANDIMMUNE™), colchicine (or COLCRYS™, MITIGARE™), felodipine (or PLENDIL™), ketoconazole (or NIZORAL™), lansoprazole (or PREVACID™), omeprazole (or LOSEC™ PRILOSEC™, ZEGERID™), nifedipine (or ADALAT™, PROCARDIA™), paroxetine (or PAXIL™, SEROXAT™), reserpine, saquinavir (or INVIRASE™, FORTOVASE™) sertraline (or ZOLOFT™), quinidine (or QUINAGLUTE™, QUINIDEX™), tamoxifen (or NOLVADEX™, GENOX™, TAMIFEN™), duloxetine (or CYMBALTA, ARICLAIM), loperamide (or IMODIUM™), loperamide (or IMODIUM™) azidothymidine (or zidovudine, RETROVIR™) or a homodimer thereof, elacridar, zosuquidar, laniquidar, valspodar, reversan, or a combination thereof;

(iii) an antihistamine, wherein optionally the antihistamine is fexofenadine (or ALLEGRA™), loratadine (or CLARITIN™), hydroxyzine (or ATARAX™) diphenhydramine (or BENADRYL™), acrivastine or cetirizine;

(iv) an ATP-binding cassette (ABC) inhibitor, wherein optionally the ABC inhibitor is a tyrosine kinase (TK) inhibitor (TKI), and optionally the TKI comprises imatinib (or GLEEVEC™, GLIVEC™), gefitinib (or IRESSA™), erlotinib (or TARCEVA™), ceritinib (or ZYKADIA™), lenvatinib (or LENVIMA™ LENVANIX™), or a small inhibitory RNA (siRNA) that inhibits an ABC or a TK; or

(v) any combination of (i) to (iv).

In alternative embodiments, provided are formulations comprising a compound or composition as provided herein, wherein optionally the formulation is a solid, liquid, gel, aerosol, powder or emulsion formulation.

In alternative embodiments, provided are pharmaceutical compositions comprising a therapeutic combination as provided herein or a formulation as provided herein, wherein optionally the pharmaceutical composition is formulated for enteral or parenteral administration.

In alternative embodiments, provided are a pump, a device, a subcutaneous infusion device, a continuous subcutaneous infusion device, an infusion pen, a needles, a reservoir, an ampoules, a vial, a syringe, a cartridge, a disposable pen or jet injector, a prefilled pen or a syringe or a cartridge, a cartridge or a disposable pen or jet injector, a two chambered or multi-chambered pump, a syringe, a cartridge or a pen or a jet injector, comprising a therapeutic combination as provided herein, or a formulation as provided herein, or a pharmaceutical composition as provided herein.

In alternative embodiments, provided are methods for treating, ameliorating or protecting (preventing) an organophosphate toxicity or poisoning or toxic exposure, or for treating, ameliorating or protecting (preventing) organophosphate inhibition of an acetylcholinesterase (AChE), comprising:

administering to a patient or an individual in need thereof, a therapeutic combination as provided herein, or a formulation as provided herein, or a pharmaceutical composition as provided herein, wherein optionally the compound or formulation is administered enterally or parenterally,

wherein optionally the compound or formulation is administered orally, sublingually, parenterally, by inhalation spray, nasally, topically, intrathecally, intrathecally, intracerebrally, epidurally, intracranially or rectally, or

administering the therapeutic combination as provided herein, or the formulation as provided herein, or the pharmaceutical composition as provided herein, using a pump, a device, a subcutaneous infusion device, a continuous subcutaneous infusion device, an infusion pen, a needles, a reservoir, an ampoules, a vial, a syringe, a cartridge, a disposable pen or jet injector, a prefilled pen or a syringe or a cartridge, a cartridge or a disposable pen or jet injector, a two chambered or multi-chambered pump, a syringe, a cartridge or a pen or a jet injector as provided herein.

In alternative embodiments of the methods: the organophosphate toxicity, poisoning or toxic exposure is caused by exposure of the patient or individual to an alkyl methylphosphonate or related nerve agent, or an alkylphosphorate insecticide, and optionally the organophosphate (OP) is or is a component of a toxin, an herbicide, an insecticide, or a nerve gas or nerve agent, and optionally the organophosphate (OP) is or comprises parathion, malathion, methyl parathion, chlorpyrifos, diazinon, dichlorvos, phosmet, fenitrothion, tetra-chlorvinphos, azamethiphos or azinphos methyl; or, the nerve agent is or comprises VX (or ethyl ({2-[bis(propan-2-yl)amino]ethyl} sulfanyl) (methyl) phosphinate), paraoxon, soman (O-Pinacolyl methylphosphonofluoridate), tabun (Ethyl N,N Dimethyl-phosphoramido-cyanidate), sarin ((RS)-propan-2-yl methyl-phosphono-fluoridate) or a combination thereof.

In alternative embodiments of the methods: the acetylcholinesterase (AChE) is in the central nerve system (CNS), or the acetylcholinesterase (AChE) is a human acetylcholinesterase (hAChE).

In alternative embodiments, provided are methods for treating, preventing or ameliorating excessive acetylcholine stimulation in the brain, comprising:

administering to a patient or an individual in need thereof, a therapeutic combination as provided herein, or a formulation as provided herein, or a pharmaceutical composition as provided herein,

wherein optionally the compound or formulation is administered enterally or parenterally,

wherein optionally the compound or formulation is administered orally, sublingually, parenterally, by inhalation spray, nasally, topically, intrathecally, intrathecally, intracerebrally, epidurally, intracranially or rectally, or

administering the therapeutic combination as provided herein, or the formulation as provided herein, or the pharmaceutical composition as provided herein, using a pump, a device, a subcutaneous infusion device, a continuous subcutaneous infusion device, an infusion pen, a needles, a reservoir, an ampoules, a vial, a syringe, a cartridge, a disposable pen or jet injector, a prefilled pen or a syringe or a cartridge, a cartridge or a disposable pen or jet injector, a two chambered or multi-chambered pump, a syringe, a cartridge or a pen or a jet injector as provided herein.

In alternative embodiments of the methods: the excessive acetylcholine stimulation in the brain, the CNS or the PNS is caused by a drug, a drug overdose, or a poisoning or a toxic exposure to a drug, and optionally the drug overdose causing the excessive acetylcholine stimulation is caused at least in part by physostigmine, neostigmine, pyridostigmine, diisopropylfluorophosphate and/or echothiophate.

In alternative embodiments, provided are uses of a therapeutic combination as provided herein, or a formulation as provided herein, or a pharmaceutical composition as provided herein, or a a pump, a device, a subcutaneous infusion device, a continuous subcutaneous infusion device, an infusion pen, a needles, a reservoir, an ampoules, a vial, a syringe, a cartridge, a disposable pen or jet injector, a prefilled pen or a syringe or a cartridge, a cartridge or a disposable pen or jet injector, a two chambered or multi-chambered pump, a syringe, a cartridge or a pen or a jet injector as provided herein, for: treating, preventing or ameliorating excessive acetylcholine stimulation in the brain; or, treating, ameliorating or protecting (preventing) an organophosphate toxicity or poisoning or toxic exposure, or for treating, ameliorating or protecting (preventing) organophosphate inhibition of an acetylcholinesterase (AChE).

In alternative embodiments, provided are methods or use of any of the preceding claims, wherein: (a) the nucleophilic hydroxyimino-acetamido alkylamine antidotes and the brain efflux transporter inhibitor and/or the inhibitor of renal tubular secretion or administered together (or substantially at the same time), and/or are formulated together; or (b) the nucleophilic hydroxyimino-acetamido alkylamine antidotes and the brain efflux transporter inhibitor and/or the inhibitor of renal tubular secretion or administered separately, and/or are formulated separately.

In alternative embodiments, provided are therapeutic combinations as provided herein, or a formulation as provided herein, or a pharmaceutical composition as provided herein, or a pump, a device, a subcutaneous infusion device, a continuous subcutaneous infusion device, an infusion pen, a needles, a reservoir, an ampoules, a vial, a syringe, a cartridge, a disposable pen or jet injector, a prefilled pen or a syringe or a cartridge, a cartridge or a disposable pen or jet injector, a two chambered or multi-chambered pump, a syringe, a cartridge or a pen or a jet injector as provided herein, for use in: treating, preventing or ameliorating excessive acetylcholine stimulation in the brain; or, treating, ameliorating or protecting (preventing) an organophosphate toxicity or poisoning or toxic exposure, or for treating, ameliorating or protecting (preventing) organophosphate inhibition of an acetylcholinesterase (AChE).

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1 illustrates a table showing RS194B and 2PAM Protective Indices upon intramuscular (IM, or i.m.) and oral (or p.o.) administration, the data showing the oral bioavailability of RS194B, and lack thereof for 2-PAM since it is a quaternary drug and will not rapidly cross the intestinal absorptive barrier or the blood-brain barrier.

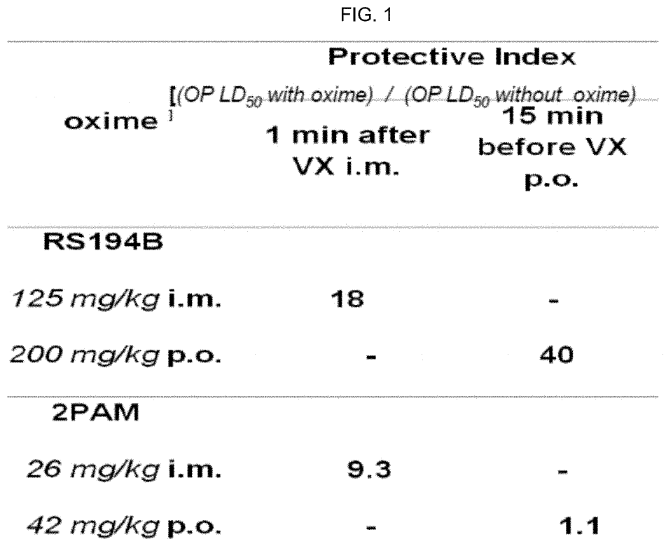

FIG. 2 schematically illustrates the dynamics of sarin going from the lung to the brain, and,

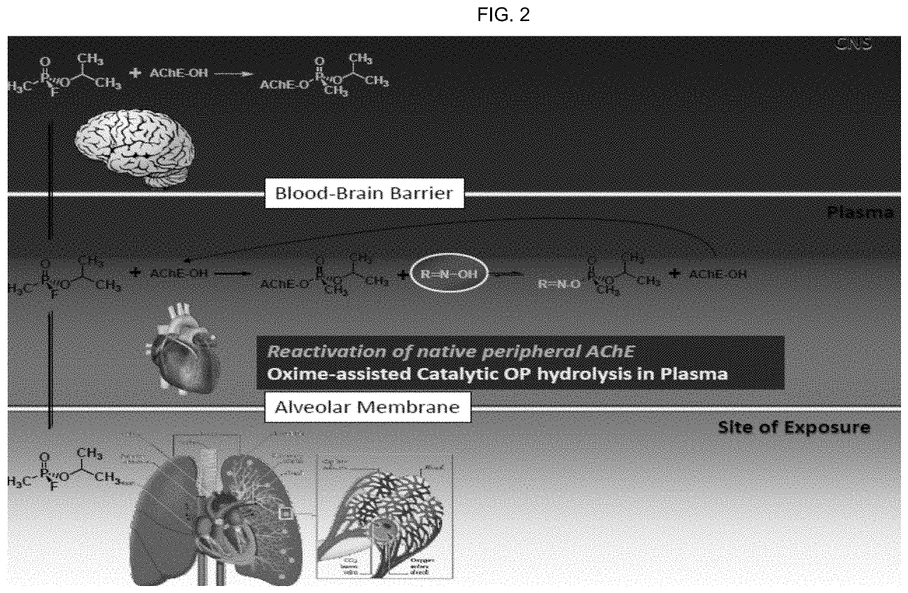

FIG. 3 schematically illustrates the dynamics of a reactivating oxime that can form a neutral (un-ionized) species to cross the blood-brain barrier.

FIG. 4 graphically illustrates the plasma levels of RS194B after intravenous (IV), intra-muscular (IM) and per-oral (p.o.) dosing in mice, data illustrates are PK profiles of RS194B in mouse plasma following various routes of administration to the mouse: i.v. −20 mg/kg; p.o., 50 mg/kg; p.o., 200 mg/kg; i.m., 80 mg/kg; the data show rapid oral absorption within 20 min and bioavailability >50%; note that: (a) systemic distribution after intramuscular administration is rapid, (b) brain concentrations after parenteral administration exceed plasma concentrations after 30 minutes.

FIG. 5 graphically illustrates plasma and brain concentrations of RS194B after an intramuscular loading dose followed by four oral maintenance doses; the data shows that plasma and brain concentrations after a loading dose and frequent maintenance dosing sustains a steady-state concentration.

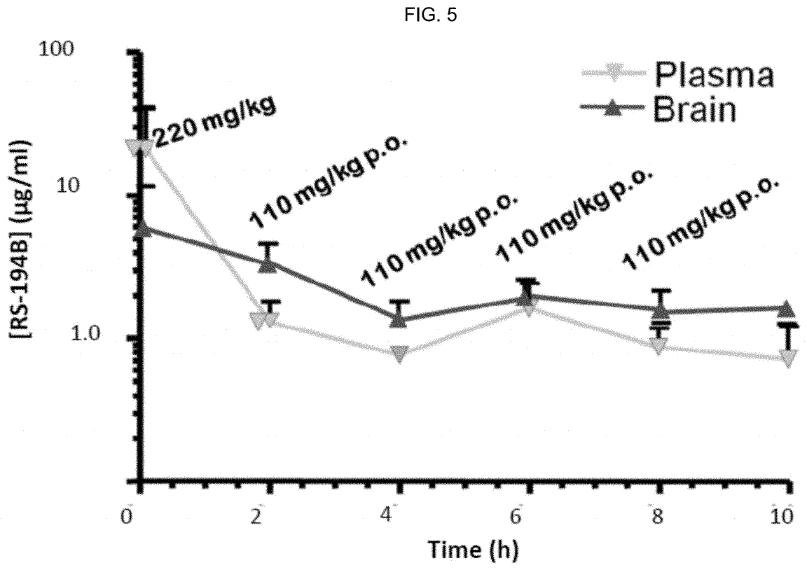

FIG. 6 graphically illustrates the recovery of macaque blood cholinesterase activities post-sarin administration; the data shows that RS194B restores plasma butyrylcholinesterase (BChE) and red blood cell acetylcholinesterase (AChE) activity in macaques when given 3 minutes post-exposure.

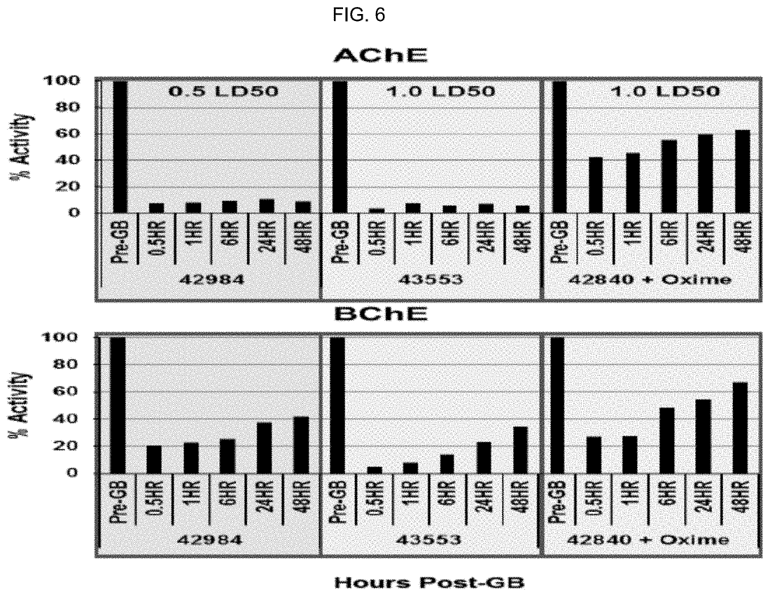

FIG. 7 graphically illustrates the recovery of macaque blood cholinesterase activities post-sarin exposure (Top graph) AChE and (Bottom graph) BChE; the oxime RS194B was administered at 62.5 mg/kg and atropine 0.28 mg/kg 2.75 min after a 31 min sarin exposure; BChEpre-administered by inhalation to obtain 6.0 mg/kg deposited; green monkeys survived, red and orange monkeys died; survival was greatly enhanced in macaques after RS194B given 3 minutes post-exposure of sarin and approximately 30 minutes after initiation of sarin exposure.

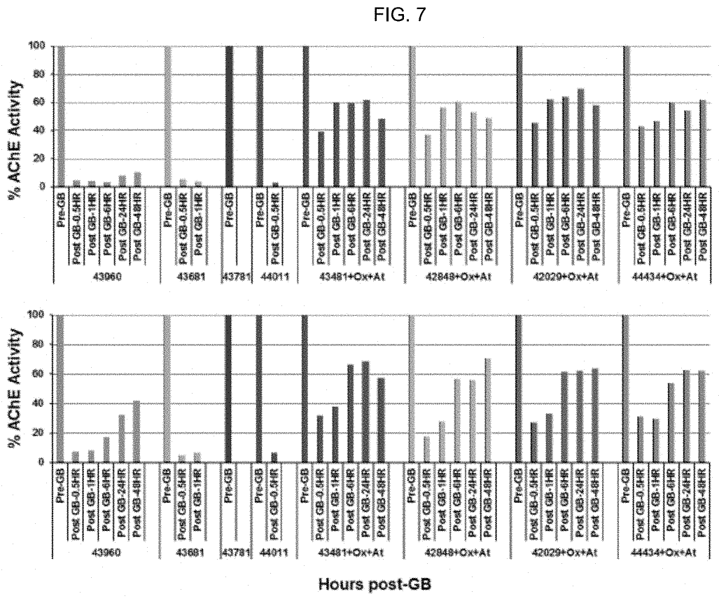

FIG. 8 graphically illustrates % AChE and BChE activities in blood pre- and post-exposure to paraoxon (PX); RS194B (70 mg/kg) was administered (IM) after termination of 30 to 40 min aerosol PX exposure, and recovery of plasma BChE and red cell AChE after paraoxon aerosol exposure; conditions are similar to those for sarin vapor; paraoxon has to be given as an aerosol since it has a lower vapor pressure.

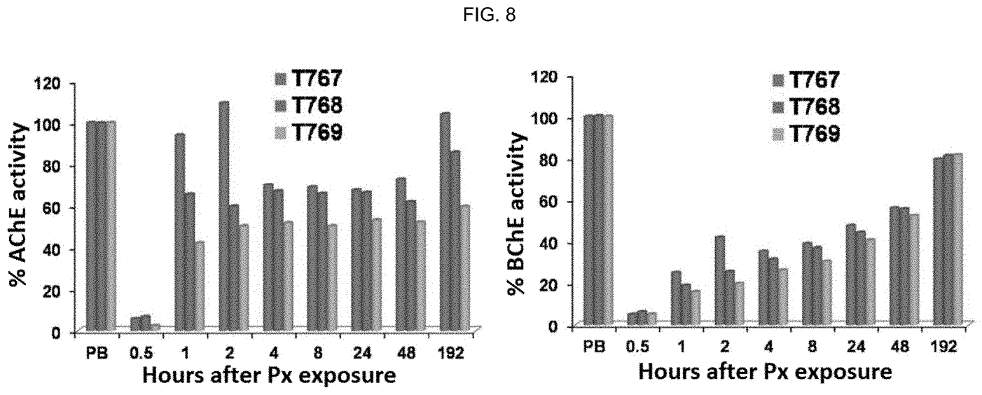

FIG. 9 graphically illustrates plasma concentrations after three routes of RS194B (70 mg/kg) administration in mice; rapid clearance of RS194B after intravenous (i.v.) tail vein and retro-orbital and intramuscular (i.m.) administration. For control mice, no organophosphate was administered.

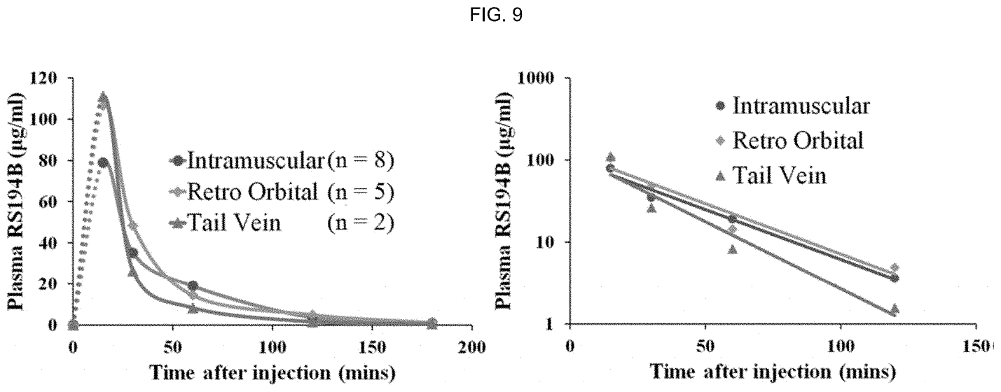

FIG. 10 schematically illustrates the fundamental functions of transporters: the short residence time of soluble, non-hydrophoblic oximes depends on renal transport from the kidney to the urine and extrusion from the brain by brain capillary endothelial transporters after the oxime crosses the blood brain barrier.

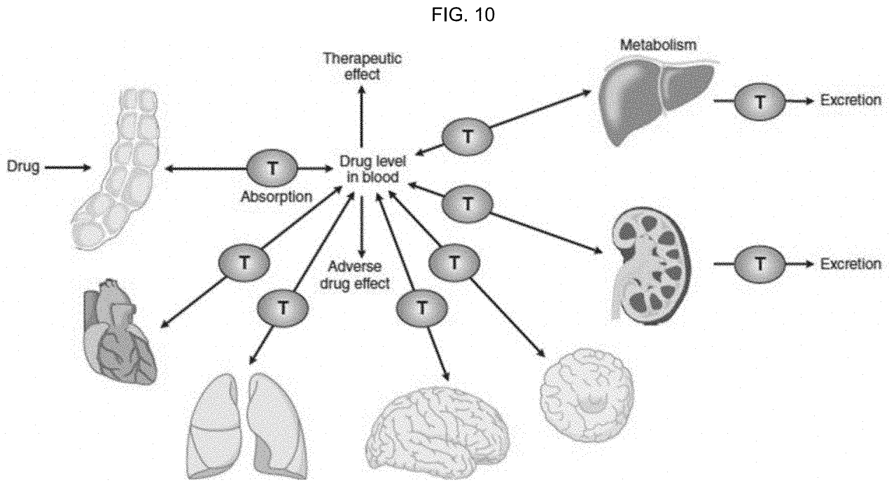

FIG. 11A-B graphically illustrates data showing the enhancement of plasma and brain levels of RS194B after administration of the P-glycoprotein inhibitor tariquidar; the graphs show drug concentration in μg/mg, [symbol] with and [symbol] without tariquidar; mice were 10 minutes (min) prior IP treated with tariquidar, then the indicated organs were collected: FIG. 11A 15 min and FIG. 11B 10 min after RS194B administration, n=3.

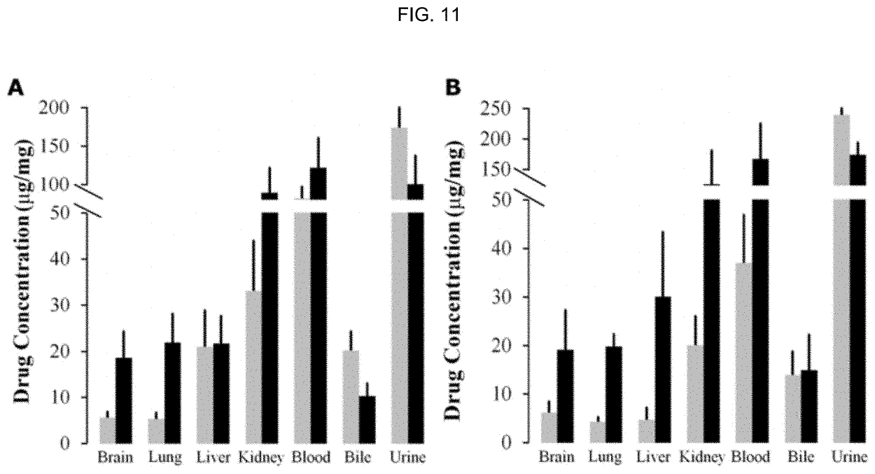

FIG. 12A-B graphically illustrates data showing enhancement of plasma, brain and other tissue levels by knocking out the P-glycoprotein gene; the data shows that brain, kidney and plasma levels are increased while urine levels are decreased; RS194B was administered IM to [symbol] the brain, [symbol] lung, [symbol] liver, [symbol] kidney, [symbol] blood, [symbol] diaphragm, [symbol] urine in FIG. 12A FVB control mice and FIG. 12B P-glycoprotein [symbol] knockout mice, n=3.

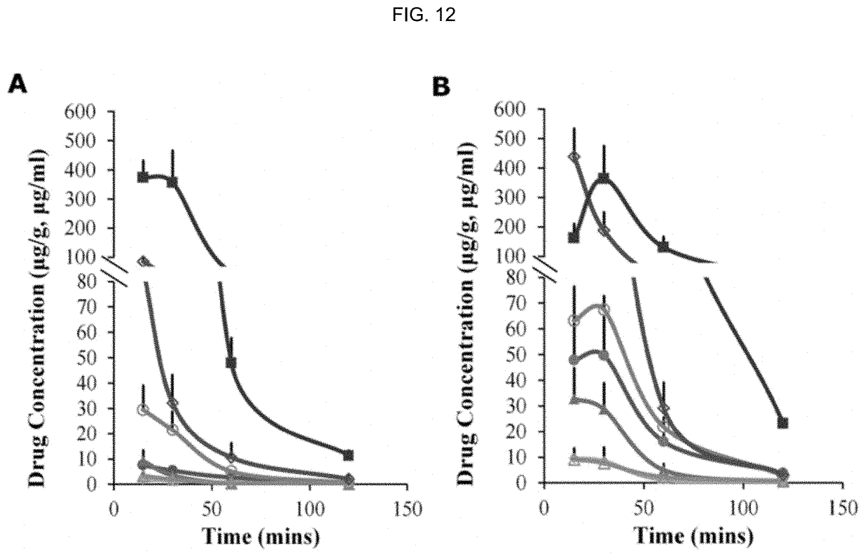

FIG. 13 graphically illustrates data showing the pharmacokinetic influence of two zwitterionic fluoroquinolines: ciprfloxacin and levofloxacin on RS194B levels.

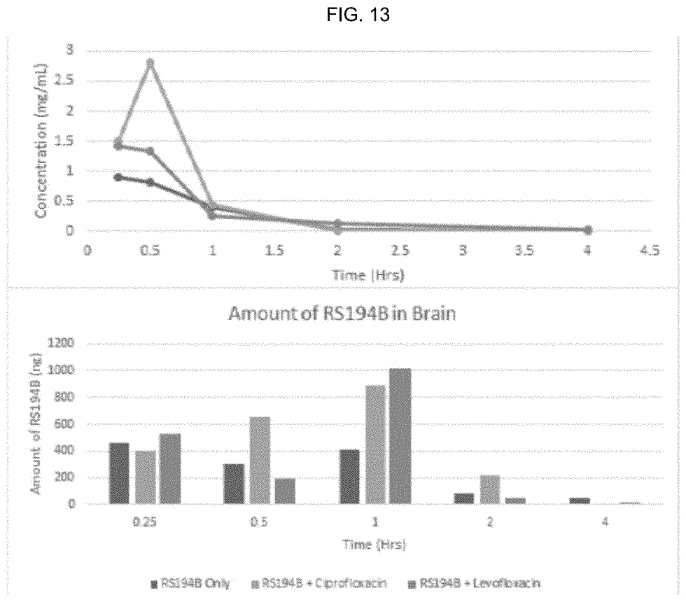

FIG. 14 graphically illustrates data showing that a greater enhancement of RS194B levels in seen with diltiazem, a Ca++ channel blocker; upper graph shows RS194B concentration in plasma, and the lower graph shows RS194B levels in the brain.

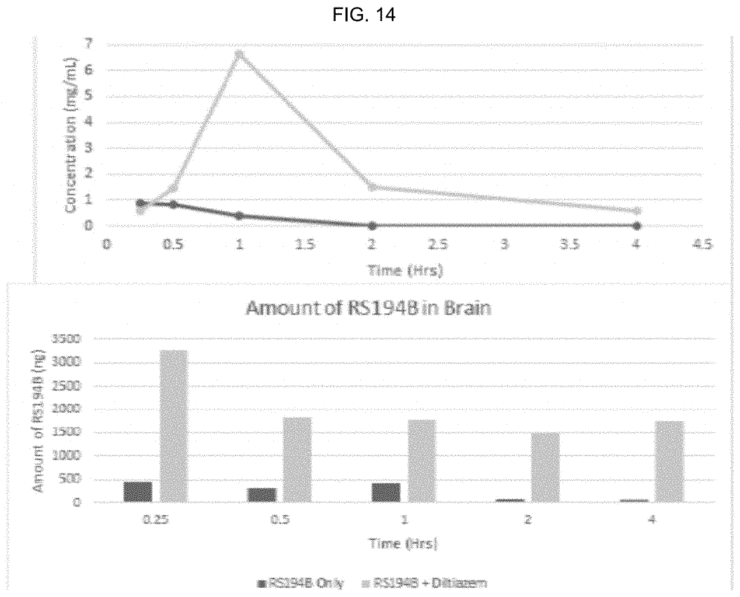

Like reference symbols in the various drawings indicate like elements.

### DETAILED DESCRIPTION

Provided are therapeutic combinations comprising: nucleophilic hydroxyimino-acetamido alkylamine antidotes that cross the blood-brain barrier (BBB) to catalyze the hydrolysis of organophosphate (OP)-inhibited human acetylcholinesterase (hAChE) in the central nervous system (CNS); and, a brain efflux transporter inhibitor, or an inhibitor of renal tubular secretion. In alternative embodiments, the nucleophilic hydroxyimino-acetamido alkylamine antidote comprises a compound as described herein, or a compound as described in U.S. patent application publication no. US2015/0361060 A1 or WO/2014/127315 A1. In alternative embodiments, the brain efflux transporter inhibitor or the inhibitor of renal tubular secretion comprises or is: (i) metformin (or GLUCOPHAGE™); a fluoroquinolone antibiotic (wherein optionally the fluoroquinolone is ciprofloxacin, or CILOXAN™, CIPRO™, or NEOFLOXIN™), norfloxacin (or NOROXIN™), levofloxacin (or LEVAQUIN™), or a combination thereof, (ii) a p-glycoprotein inhibitor, wherein optionally the p-glycoprotein inhibitor is or comprises: tariquidar, clarithromycin (or BIAXIN™), erythromycin (or ERYTHROCIN™), ritonavir (or NORVIR™), verapamil (or CALAN™, COVERA™, VERELAN™, TARKA™) gallopamil (or methoxyverapamil), dimeditiapramine (or tiapamil), diltiazem (or CARDIZEM™), amiodarone (or CORDARONE™, NEXTERONE™), ciclosporin (or NEORAL™, SANDIMMUNE™), colchicine (or COLCRYS™, MITIGARE™) felodipine (or PLENDIL™), ketoconazole (or NIZORAL™), lansoprazole (or PREVACID™), omeprazole (or LOSEC™, PRILOSEC™, ZEGERID™), nifedipine (or ADALAT™, PROCARDIA™), paroxetine (or PAXIL™, SEROXAT™), reserpine, saquinavir (or INVIRASE™, FORTOVASE™), sertraline (or ZOLOFT™), quinidine (or QUINAGLUTE™, QUINIDEX™), tamoxifen (or NOLVADEX™, GENOX™ TAMIFEN™), duloxetine (or CYMBALTA, ARICLAIM), loperamide (or IMODIUM™), azidothymidine (or zidovudine, RETROVIR™) or a homodimer thereof (see e.g., Namanja-Magliano et al (2017) Bioorganic & Medicinal Chem vol 25(19):5128-5132), elacridar, zosuquidar, laniquidar, valspodar, reversan (see e.g., Burkhart et al Cancer Res. (2009) vol 69(16):6573-80), or a combination thereof; (iii) an antihistamine, wherein optionally the antihistamine is fexofenadine (or ALLEGRA™) loratadine (or CLARITIN™), hydroxyzine (or ATARAX™), diphenhydramine (or BENADRYL™), acrivastine or cetirizine; (iv) an ATP-binding cassette (ABC) inhibitor, wherein optionally the ABC inhibitor is a tyrosine kinase (TK) inhibitor (TKI), and optionally the TKI comprises imatinib (or GLEEVEC™, GLIVEC™), gefitinib (or IRESSA™), erlotinib (or TARCEVA™), ceritinib (or ZYKADIA™), lenvatinib (or LENVIMA™, LENVANIX™), or a small inhibitory RNA (siRNA) that inhibits an ABC or a TK; or, (v) any combination of (i) to (iv).

In alternative embodiments, the nucleophilic hydroxyimino-acetamido alkylamine antidotes used in the therapeutic combinations are provided herein (e.g., RS194B or RS138B) have improved in vivo functionality because they are combined with or used with a brain efflux transporter inhibitor, or an inhibitor of renal tubular secretion. In alternative embodiments, the nucleophilic hydroxyimino-acetamido alkylamine antidotes used in the therapeutic combinations because they are combined with or used with a brain efflux transporter inhibitor, or an inhibitor of renal tubular secretion, have enhanced retention in the brain, diminished secretion from renal tubules, and/or slowed in vivo clearance; this has been demonstrated in a mouse model.

In alternative embodiments, the nucleophilic hydroxyimino-acetamido alkylamine antidotes used in the therapeutic combinations are provided herein (e.g., RS194B or RS138B) have a prolonged duration of action in vivo and enhanced concentrations are retained in the central nervous system, including the brain.

In alternative embodiments, therapeutic combinations are provided herein are an improved antidote to organophosphate poisoning, e.g., as initiated from terrorism or excessive pesticide use, where a diminished dose and frequency of dosing is required. Given the known characteristics of the most dangerous and volatile organophosphates, this invention focuses on the following characteristics: a) pulmonary exposure as the primary portal of toxicity; and, b) short term rapid recovery of toxicity through a loading dose of antidote, followed by a maintenance dose to sustain antidote action.

In alternative embodiments, use of therapeutic combinations are provided herein allows administration of lower dose of nucleophilic hydroxyimino-acetamido alkylamine antidote, e.g., RS194B, and prolongs its action, by co-formulation and\or co-administration of the brain efflux transporter inhibitor or the inhibitor of renal tubular secretion.

In alternative embodiments, the brain efflux transporter inhibitor or the inhibitor of renal tubular secretion are known compounds used therapeutically that have safety profiles with the FDA; hence the brain efflux transporter inhibitor or the inhibitor of renal tubular secretion can be an FDA approved agent, e.g., such as metformin or a fluoroquinolone.

For example, in alternative embodiments, the dose range for RS 194B is between about 30 and 80 mgs/kg, but with the brain efflux transporter inhibitor or the inhibitor of renal tubular secretion (e.g., a repurposed agent), the administered dose can go down considerably in dose and frequency because the efflux transport out of the brain and/or renal tubular secretion is inhibited or blocked. In alternative embodiments, tubular secretion clears the agent from the body.

In alternative embodiments, therapeutic combinations as provided herein can: cause inhibition of brain efflux, thus promoting retention in the brain where the target acetylcholinesterase resides; and/or cause inhibition of renal tubular secretion, thus causing retention and slowing the pharmacokinetics of elimination. In alternative embodiments, antidote component of the therapeutic combination as provided herein is eliminated unchanged largely by the kidney, i.e., it is not appreciably metabolized.

In alternative embodiments, the hydroxyimino-acetamido alkylamines as used in the therapeutic combinations as provided herein are designed to fit within AChE gorge dimensions and interact with the conjugated phosphate atom in the gorge. In alternative embodiments, the hydroxyimino-acetamido alkylamines as provided herein are designed for optimized fit within the gorge and ionization equilibria that govern affinity and reactivity for the two linked hAChE re-activation steps. In alternative embodiments, compositions used in therapeutic combinations as provided herein comprise uncharged but ionizable N-substituted 2-hydroxyimino-acetamido alkylamine reactivators of phosphorylated human acetylcholinesterase (hAChE) intended to catalyze the hydrolysis of organophosphate (OP)-inhibited hAChE in the CNS. In alternative embodiments, compositions used in therapeutic combinations as provided herein, or used to practice methods as provided herein, comprise the so-called RS194B, an azepine analog, and RS2-138B structures and compounds.

In alternative embodiments, the terminal alkylamine is attached to an aminocabonylaldoxime or an alkylketo-oxine forming a bis-functional nucleophile, as reported previously for the pyridinium aldoxime.

In alternative embodiments, compositions used in therapeutic combinations as provided herein are rapidly absorbed from the site of administration (e.g., oral, inhalation, or intramuscular, or i.m.), cross the blood-brain barrier as a neutral species, show enhanced nucleophicity from the oximate ionization species, selectively form a reversible complex with the active center gorge of an AChE, displace the covalently attached OPs, e.g., from a organophosphate toxicant such as a pesticide or a nerve agent, and restore AChE activity in the brain and periphery. In alternative embodiments, compositions used in therapeutic combinations as provided herein provide immediate protection from exposure, as well as prevention, of OP exposure, e.g., protection or prevention of immediate and recurring seizures that result from excessive acetylcholine stimulation in the brain.

With these exemplary properties, therapeutic combinations as provided herein can access the CNS and peripheral nervous system and reactivate cholinesterases inhibited by pesticides and nerve agents. In alternative embodiments, therapeutic combinations as provided herein can be an antidote for poisoning by organophosphate (e.g., diisopropylfluorophosphates and echothiophate) and carbamylating drugs (e.g., physostigmine, neostigmine and pyridostigmine). In alternative embodiments, therapeutic combinations as provided herein can assist in the catalysis of the organophosphate itself before it gets to the enzyme; hence, compositions of the invention can have a scavenging capacity for the parent organophosphate; catalysis is carried out by acetylcholinesterase or butyrylcholinesterase and the oxime facilitates this.

In alternative embodiments, therapeutic combinations as provided herein can not only cross the blood-brain barrier to reactivate acetylcholinesterase in the CNS and peripheral nervous system, but compounds of the invention also are effective as antidotes and protective (prophylactic) agents when given orally, e.g., sublingually. In alternative embodiments, for example, as a prophylactic agent, the oral dosage route does not have the most rapid onset, but is an ideal route for achieving prolonged antidote activity; this can be the most practical route for prophylactic use against massive releases of volatile gases, for example, prophylactic or therapeutic use in accidents or in poison gas (e.g., nerve agent) warfare, e.g., paraoxon, sarin, cyclosarin and VX attacks.

In alternative embodiments, therapeutic combinations as provided herein have improved antidote efficacy for various organophosphates, e.g., methylphosphonate nerve agents and phosphorate insecticides, and have a lower toxicity than 2-PAM (pralidoxime, or 2-pyridine aldoxime methyl chloride), and an increased half-life in the brain when compared with other compounds. In alternative embodiments, therapeutic combinations as provided herein, or compounds used in methods as provided herein, e.g., the compound RS194B, are designed to have an optimized fit within an AChE gorge, and ionization equilibria dictating affinity and reactivity for the two linked reactivation steps.

In alternative embodiments, transport inhibitor given at a single dose can enhance brain concentrations and reduce body clearance of RS194B; physiologic studies provide support for this capability by showing that: no detectable metabolism of RS194B and analogues was found in microsomal fractions from liver of primate and non-primate mammals; studies with tariquidar, a transport inhibitor, show enhanced brain, kidney and liver concentrations of RS194B, with concomitant reductions of urine and bile concentrations in mice; studies in a P-glycoprotein deleted mouse strain (mdr 1a/b), show enhanced brain, kidney and liver concentrations of RS194B; and, a screen of FDA approved drugs show little change in RS 194B levels in tissues and urine with metformin, but increases with two fluoroquinolones, ciprofloxacin and levofloxacin, and the Ca⁺⁺ channel blocker, diltiazem.

### Products of Manufacture, Kits

In alternative embodiments, provided are products of manufacture and kits for practicing the methods as provided herein. In alternative embodiments, provided are products of manufacture and kits comprising all the components needed to practice a method as provided herein. In alternative embodiments, provided are kits comprising therapeutic combinations as provided herein, or compounds used to practice methods as provided herein.

In alternative embodiments, provided are kits comprising compositions and/or instructions for practicing methods as provided herein. In alternative embodiments, kits as provided herein comprise: a therapeutic combination as provided herein and instructions for use thereof.

In alternative embodiments, provided are pumps, devices, subcutaneous infusion devices, continuous subcutaneous infusion device, infusion pens, needles, reservoirs, ampoules, vials, syringes, cartridges, disposable pen or jet injectors, prefilled pens or syringes or cartridges, cartridge or disposable pen or jet injectors, two chambered or multi-chambered pumps, syringes, cartridges or pens or jet injectors comprising a composition or a formulation of the invention. In alternative embodiments, the injector is an autoinjector, e.g., a SMARTJECT® autoinjector (Janssen Research and Development LLC); or a MOLLY®, or DAI®, or DAI-RNS® autoinjector (SHL Group, Deerfield Beach, Fla.). In alternative embodiments, the injector is a hypodermic or a piston syringe.

### Formulations and Pharmaceutical Compositions

In alternative embodiments, provided are therapeutic combinations, including formulations and pharmaceutical compositions, for use in in vivo, in vitro or ex vivo methods for catalyzing the hydrolysis of organophosphate (OP)-inhibited human acetylcholinesterase (hAChE) in the central nerve system (CNS); or, for treating, ameliorating or protecting (preventing) an organophosphate toxicity or poisoning or toxic exposure, or for treating, ameliorating or protecting (preventing) organophosphate inhibition of an acetylcholinesterase (AChE); or, for treating, preventing or ameliorating excessive acetylcholine stimulation in the CNS, or the brain.

In alternative embodiments, the pharmaceutical compositions of the invention can be administered parenterally, topically, orally or by local administration, such as by aerosol (in alternative embodiments using, e.g., liposomes, powders) or transdermally (in alternative embodiments using, e.g., gels or liquids). In alternative embodiments, pharmaceutical compositions can be prepared in various forms, such as granules, tablets, pills, capsules, suspensions, taken orally (e.g., sublingually), suppositories and salves, lotions and the like. In alternative embodiments, the therapeutic combinations, formulations and pharmaceutical compositions as provided herein comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, geltabs, on patches, in implants, etc.

In alternative embodiments, the therapeutic combinations, formulations and pharmaceutical compositions as provided herein can be delivered by transdermally, by a topical route, formulated as applicator sticks, solutions, suspensions, emulsions, gels, creams, ointments, pastes, jellies, paints, powders, and aerosols. Oral carriers can be elixirs, syrups, capsules, tablets, pills, geltabs and the like.

In alternative embodiments, therapeutic combinations, formulations and pharmaceutical compositions as provided herein are delivered orally (e.g., sublingually), e.g., as pharmaceutical formulations for oral administration, and can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

In alternative embodiments, liquid carriers are used to manufacture or formulate therapeutic combinations, formulations and pharmaceutical compositions as provided herein, or a therapeutic combinations, formulations and pharmaceutical compositions as provided herein used to practice the methods as provided herein, including carriers for preparing solutions, suspensions, emulsions, syrups, elixirs and pressurized compounds. The active ingredient (e.g., a therapeutic combinations, formulations and pharmaceutical compositions as provided herein) can be dissolved or suspended in a pharmaceutically acceptable liquid carrier such as water, an organic solvent, a mixture of both or pharmaceutically acceptable oils or fats. The liquid carrier can comprise other suitable pharmaceutical additives such as solubilizers, emulsifiers, buffers, preservatives, sweeteners, flavoring agents, suspending agents, thickening agents, colors, viscosity regulators, stabilizers or osmo-regulators.

In alternative embodiments, solid carriers are used to manufacture or formulate therapeutic combinations, formulations and pharmaceutical compositions as provided herein, or a therapeutic combinations, formulations and pharmaceutical compositions as provided herein used to practice the methods as provided herein, including solid carriers comprising substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

In alternative embodiments, concentrations of therapeutically active compound in therapeutic combinations, formulations and pharmaceutical compositions as provided herein can be from between about 0.1% to about 100% by weight.

In alternative embodiments, therapeutic combinations, formulations and pharmaceutical compositions as provided herein are prepared by any method well known in the art, e.g., as described by Brunton et al., eds., Goodman and Gilman's: The Pharmacological Bases of Therapeutics, 12th ed., McGraw-Hill, 2011; Remington: The Science and Practice of Pharmacy, Mack Publishing Co., 20th ed., 2000; Avis et al., eds., Pharmaceutical Dosage Forms: Parenteral Medications, published by Marcel Dekker, Inc., N.Y., 1993; Lieberman et al., eds., Pharmaceutical Dosage Forms: Tablets, published by Marcel Dekker, Inc., N.Y., 1990; and Lieberman et al., eds., Pharmaceutical Dosage Forms: Disperse Systems, published by Marcel Dekker, Inc., N.Y., 1990.

In alternative embodiments, therapeutic combinations, formulations and pharmaceutical compositions as provided herein are delivered by any effective means appropriated for a particular treatment. For example, depending on the specific agent to be administered, the suitable means include oral, rectal, vaginal, nasal, pulmonary administration, or parenteral (including subcutaneous, intramuscular, intravenous and intradermal) infusion into the bloodstream. For parenteral administration, therapeutic combinations, formulations and pharmaceutical compositions as provided herein may be formulated in a variety of ways. Aqueous solutions of the modulators can be encapsulated in polymeric beads, liposomes, nanoparticles or other injectable depot formulations known to those of skill in the art. In alternative embodiments, therapeutic combinations, formulations and pharmaceutical compositions as provided herein are administered encapsulated in liposomes (see below). In alternative embodiments, depending upon solubility, therapeutic combinations, formulations and pharmaceutical compositions as provided herein are formulated in an aqueous layer and in a lipidic layer, e.g., a liposomic suspension. In alternative embodiments, a hydrophobic layer comprises phospholipids such as lecithin and sphingomyelin, steroids such as cholesterol, more or less ionic surfactants such a diacetylphosphate, stearylamine, or phosphatidic acid, and/or other materials of a hydrophobic nature.

In alternative embodiments, therapeutic combinations, formulations and pharmaceutical compositions as provided herein can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co., Easton Pa. (“Remington's”). For example, in alternative embodiments, therapeutic combinations, formulations and pharmaceutical compositions as provided herein are formulated in a buffer, in a saline solution, in a powder, an emulsion, in a vesicle, in a liposome, in a nanoparticle, in a nanolipoparticle and the like. In alternative embodiments, the therapeutic combinations, formulations and pharmaceutical compositions as provided herein are formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice embodiments as provided herein can be in forms such as tablets, pills, powders, capsules, liquids, gels, syrups, slurries, suspensions, etc., suitable for in vivo, in vitro or ex vivo applications.

In practicing methods and therapeutic combinations and formulations as provided herein, administered compounds (e.g., formulations) can comprise a solution of compositions (e.g., apratoxin F and apratoxin G compounds, and/or apratoxin F and apratoxin G stereoisomers, derivatives and analogs) disposed in or dissolved in a pharmaceutically acceptable carrier, e.g., acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations are sterile and can be manufactured to be generally free of undesirable matter. In one embodiment, these solutions and formulations are sterilized by conventional, well known sterilization techniques.

The solutions and formulations used to practice embodiments as provided herein can comprise auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

In alternative embodiments, therapeutic combinations and formulations as provided herein are delivered by the use of liposomes. In alternative embodiments, by using liposomes, particularly where the liposome surface carries ligands specific for target cells or organs, or are otherwise preferentially directed to a specific tissue or organ type, one can focus the delivery of an active agent into a target cells in an in vivo, in vitro or ex vivo application.

In alternative embodiments, therapeutic combinations and formulations as provided herein can be directly administered, e.g., under sterile conditions, to an individual (e.g., a patient, or an animal) to be treated. The modulators can be administered alone or as the active ingredient of a pharmaceutical composition.

In alternative embodiments, therapeutic combinations and formulations as provided herein are combined with or used in association with other therapeutic agents. For example, an individual may be treated concurrently with various conventional therapeutic agents and therapeutic combinations and formulations as provided herein.

### Nanoparticles, Nanolipoparticles and Liposomes

In alternative embodiments, provided are nanoparticles, nanolipoparticles, vesicles and liposomal membranes comprising therapeutic combinations as provided herein, e.g., for use in methods for catalyzing the hydrolysis of organophosphate (OP)-inhibited human acetylcholinesterase (hAChE) in the central nerve system (CNS); or, for treating, ameliorating or protecting (preventing) an organophosphate toxicity or poisoning or toxic exposure, or for treating, ameliorating or protecting (preventing) organophosphate inhibition of an acetylcholinesterase (AChE); or, for treating, preventing or ameliorating excessive acetylcholine stimulation in the CNS, or the brain.

Also provided are multilayered liposomes e.g., as described in Park, et al., U.S. Pat. Pub. No. 20070082042, comprising therapeutic combinations as provided herein. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice this invention.

Liposomes can be made using any method, e.g., as described in Park, et al., U.S. Pat. Pub. No. 20070042031, including method of producing a liposome by encapsulating an active agent (e.g., therapeutic combinations as provided herein), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice embodiments as provided herein comprise a substituted ammonium and/or polyanions, e.g., for targeting delivery of a therapeutic combination as provided herein to a desired cell type or organ, e.g., brain, as described e.g., in U.S. Pat. Pub. No. 20070110798.

Also provided are nanoparticles comprising therapeutic combinations as provided herein in the form of active agent-containing nanoparticles (e.g., a secondary nanoparticle), as described, e.g., in U.S. Pat. Pub. No. 20070077286. In one embodiment, the invention provides nanoparticles comprising an active agent with a bivalent or trivalent metal salt.

In one embodiment, solid lipid suspensions can be used to formulate and to deliver therapeutic combinations as provided herein in vivo, in vitro or ex vivo, as described, e.g., in U.S. Pat. Pub. No. 20050136121.

### Delivery Vehicles

In alternative embodiments, any delivery vehicle can be used to practice the methods or used to practice embodiments as provided herein, e.g., to deliver therapeutic combinations as provided herein in vivo, in vitro or ex vivo. For example, delivery vehicles comprising polycations, cationic polymers and/or cationic peptides, such as polyethyleneimine derivatives, can be used e.g. as described, e.g., in U.S. Pat. Pub. No. 20060083737.

In one embodiment, a dried polypeptide-surfactant complex, e.g. as described, e.g., in U.S. Pat. Pub. No. 20040151766, is used to formulate a therapeutic combinations as provided herein.

In one embodiment, a composition used to practice this invention can be applied to cells using vehicles with cell membrane-permeant peptide conjugates, e.g., as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, e.g., as described in U.S. Pat. No. 5,846,743, describing transport-mediating peptides that are highly basic and bind to poly-phosphoinositides.

In one embodiment, electro-permeabilization is used as a primary or adjunctive means to deliver the composition to a cell, e.g., using any electroporation system as described e.g. in U.S. Pat. Nos. 7,109,034; 6,261,815; 5,874,268.

### Dosaging

The therapeutic combinations, including pharmaceutical compositions and formulations, as provided herein can be administered for prophylactic and/or therapeutic treatments. In therapeutic applications, compositions are administered to a subject already exposed to a toxin, or exposed to any agent or chemical causing or resulting in excessive acetylcholine stimulation in the brain, e.g., exposure to a drug, a drug overdose, or a poisoning or a toxic exposure to a drug, and optionally the drug overdose causing the excessive acetylcholine stimulation is caused at least in part by: physostigmine, neostigmine, pyridostigmine, diisopropylfluorophosphate, or echothiophate an amount sufficient to cure, alleviate or partially arrest the clinical manifestations of the agent and/or its complications (a “therapeutically effective amount”).

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

For example, in alternative embodiments, administration of therapeutic combinations as provided herein comprises administration of dosages of brain efflux transporter inhibitor or inhibitor of renal tubular secretion, which can comprise: (i) metformin (or GLUCOPHAGE™); a fluoroquinolone antibiotic (wherein optionally the fluoroquinolone is ciprofloxacin, or CILOXAN™, CIPRO™, or NEOFLOXIN™) norfloxacin (or NOROXIN™), levofloxacin (or LEVAQUIN™), or a combination thereof, (ii) a p-glycoprotein inhibitor, wherein optionally the p-glycoprotein inhibitor is or comprises: tariquidar, clarithromycin (or BIAXIN™), erythromycin (or ERYTHROCIN™), ritonavir (or NORVIR™), verapamil (or CALAN™, COVERA™ VERELAN™, TARKA™), gallopamil (or methoxyverapamil), dimeditiapramine (or tiapamil), diltiazem (or CARDIZEM™), amiodarone (or CORDARONE™ NEXTERONE™), ciclosporin (or NEORAL™, SANDIMMUNE™), colchicine (or COLCRYS™, MITIGARE™), felodipine (or PLENDIL™), ketoconazole (or NIZORAL™), lansoprazole (or PREVACID™), omeprazole (or LOSEC™ PRILOSEC™, ZEGERID™), nifedipine (or ADALAT™, PROCARDIA™), paroxetine (or PAXIL™, SEROXAT™), reserpine, saquinavir (or INVIRASE™, FORTOVASE™) sertraline (or ZOLOFT™), quinidine (or QUINAGLUTE™, QUINIDEX™), tamoxifen (or NOLVADEX™, GENOX™, TAMIFEN™), duloxetine (or CYMBALTA, ARICLAIM), loperamide (or IMODIUM™), azidothymidine (or zidovudine, RETROVIR™) or a homodimer thereof, elacridar, zosuquidar, laniquidar, valspodar, reversan, or a combination thereof; (iii) an antihistamine, wherein optionally the antihistamine is fexofenadine (or ALLEGRA™), loratadine (or CLARITIN™) hydroxyzine (or ATARAX™), diphenhydramine (or BENADRYL™), acrivastine or cetirizine; (iv) an ATP-binding cassette (ABC) inhibitor, wherein optionally the ABC inhibitor is a tyrosine kinase (TK) inhibitor (TKI), and optionally the TKI comprises imatinib (or GLEEVEC™, GLIVEC™), gefitinib (or IRESSA™), erlotinib (or TARCEVA™), ceritinib (or ZYKADIA™), lenvatinib (or LENVIMA™ LENVANIX™), or a small inhibitory RNA (siRNA) that inhibits an ABC or a TK; or, (v) any combination of (i) to (iv).

In alternative embodiments, the dosages of these EDA approved drugs is the same or equivalent to known and approved dosages, for example, metaformin or ciprofloxacin can at a dosage of between about 1 to 2 grams.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention.

A number of embodiments of the invention have been described. Nevertheless, it will be understood that various modifications may be made without departing from the spirit and scope of the invention. Accordingly, other embodiments are within the scope of the following claims.

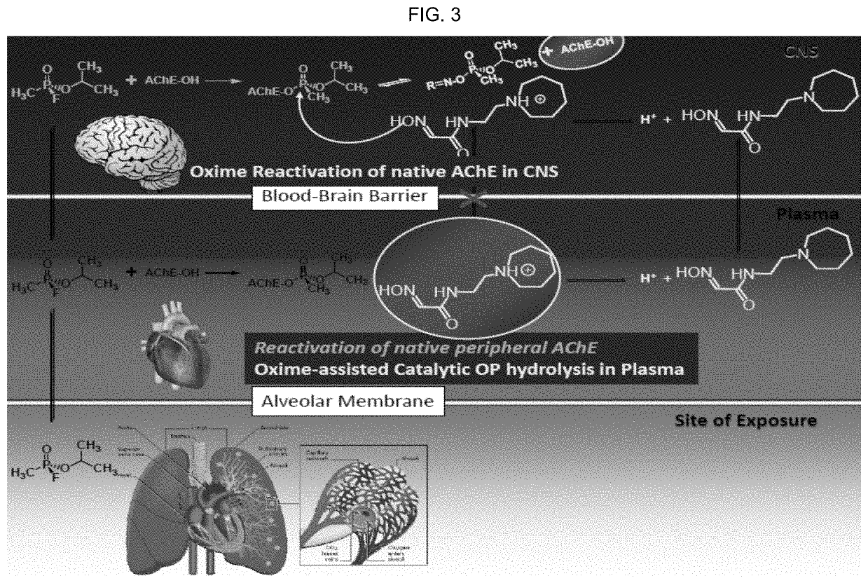

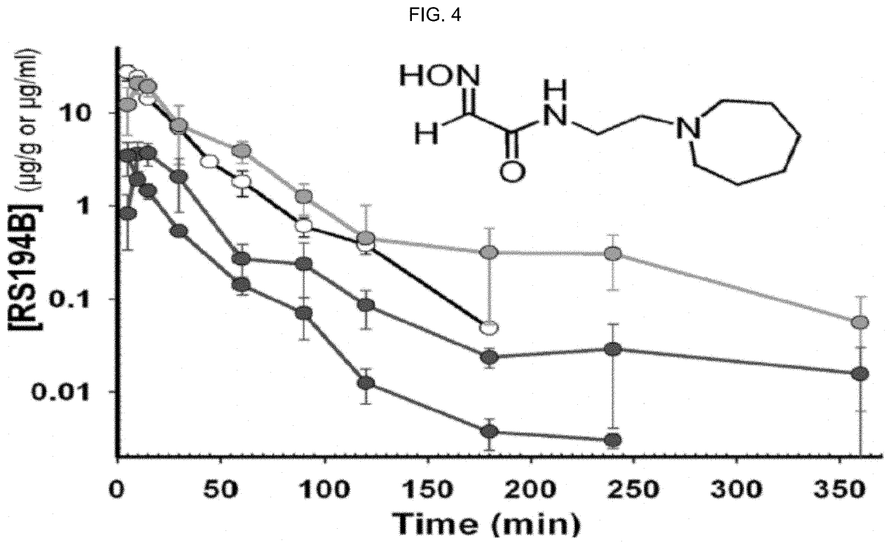

1: A therapeutic combination comprising:
(a) (i) a compound as described in U.S. patent application publication no. US2015/0361060 A1 or WO/2014/127315 A1;
(ii) a compound having one of the following structures or compositions having one or more compounds of the following structures, or equivalents thereof, or a stereoisomer thereof, or an analog thereof, or a pharmaceutically acceptable salt thereof, or a bioisostere thereof: and/or,
(iii) a composition comprising an isolated compound consisting essentially of:
(1) a compound having the formula: 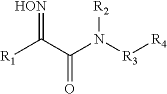
wherein:
R₁ is selected from the group consisting of: —H, -alkyl, and -aryl;
R₂ is selected from the group consisting of: —H, -alkyl, and -aryl;
R₃ is selected from the group consisting of: —(CH₂)ₙ—, and (CH₂)ₙ—CH(CH₃)—, wherein n is the integer 0, 1, 2, 3, 4 or 5;
R₄ is selected from the group consisting of: —H, -alkyl (wherein optionally the alkyl is selected from the group consisting of: -methyl, -ethyl, -propyl, -butyl, -i-propyl, and -i-butyl), -cycloalkyl (wherein optionally the cycloalkyl is selected from the group consisting of: -cyclopropyl, -cyclobutyl, -cyclopentyl, -cyclohexyl, -cycloheptyl, and -cyclooctyl), -aryl (wherein optionally the aryl is selected from the group consisting of: -phenyl, -naphthyl, -thienyl, and -indolyl), -saturated and nonsaturated heterocyclics (wherein optionally the saturated or nonsaturated heterocyclic is selected from the group consisting of: -aziridine, -oxirane-thiirane, -azirine, -oxirene, -thiirene, -azetidine, -oxetane, -thietane, -azete, -oxete, -thiete, -pyrrolidine, -oxolane, -thiolane, -pyrrole, -furan, -thiophene, -piperidine, -oxane, -thiane, -pyridine, -pyran, -thiopyran, -azepane, -oxepane, -thiepane, -azepine, -oxepine, -thiepine, -azocane, and -azocine), and bridged compounds (wherein optionally the bridged compound is selected from the group consisting of: -adamantanes, -amantadines, -biperidenes, -memantines, -methenamines, -rimantadines, -norbornanes, and -triazoles);
(2) a compound having the formula: 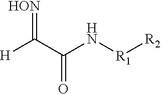
wherein
R₁ is selected from the group consisting of: —(CH₂)ₙ—, and (CH₂)ₙ—CH(CH₃)—, wherein n is the integer 0, 1, 2, 3, 4 or 5;
R₂ is selected from the group consisting of: —H, -alkyl (wherein optionally the alkyl is selected from the group consisting of: -methyl, -ethyl, -propyl, -butyl, -i-propyl, and -i-butyl), -cycloalkyl (wherein optionally the cycloalkyl is selected from the group consisting of: -cyclopropyl, -cyclobutyl, -cyclopentyl, -cyclohexyl, -cycloheptyl, and -cyclooctyl), -aryl (wherein optionally the aryl is selected from the group consisting of: -phenyl, -naphthyl, -thienyl, and -indolyl), -saturated and nonsaturated heterocyclics (wherein optionally the saturated or nonsaturated heterocyclic is selected from the group consisting of: -aziridine, -oxirane-thiirane, -azirine, -oxirene, -thiirene, -azetidine, -oxetane, -thietane, -azete, -oxete, -thiete, -pyrrolidine, -oxolane, -thiolane, -pyrrole, -furan, -thiophene, -piperidine, -oxane, -thiane, -pyridine, -pyran, -thiopyran, -azepane, -oxepane, -thiepane, -azepine, -oxepine, -thiepine, -azocane, and -azocine), and bridged compounds (wherein optionally the bridged compound is selected from the group consisting of: -adamantanes, -amantadines, -biperidenes, -memantines, -methenamines, -rimantadines, -norbornanes, and -triazoles);
(3) a compound having the formula: 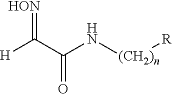
wherein
N is the integer 0, 1, 2, 3, 4, 5 or 6;
R is selected from the group consisting of: —H, -alkyl (wherein optionally the alkyl is selected from the group consisting of: -methyl, -ethyl, -propyl, -butyl, -i-propyl, and -i-butyl), -cycloalkyl (wherein optionally the cycloalkyl is selected from the group consisting of: -cyclopropyl, -cyclobutyl, -cyclopentyl, -cyclohexyl, -cycloheptyl, and -cyclooctyl), -aryl (wherein optionally the aryl is selected from the group consisting of: -phenyl, -naphthyl, -thienyl, and -indolyl), -saturated and nonsaturated heterocyclics (wherein optionally the saturated or nonsaturated heterocyclic is selected from the group consisting of: -aziridine, -oxirane-thiirane, -azirine, -oxirene, -thiirene, -azetidine, -oxetane, -thietane, -azete, -oxete, -thiete, -pyrrolidine, -oxolane, -thiolane, -pyrrole, -furan, -thiophene, -piperidine, -oxane, -thiane, -pyridine, -pyran, -thiopyran, -azepane, -oxepane, -thiepane, -azepine, -oxepine, -thiepine, -azocane, and -azocine), and bridged compounds (wherein optionally the bridged compound is selected from the group consisting of: -adamantanes, -amantadines, -biperidenes, -memantines, -methenamines, -rimantadines, -norbornanes, and -triazoles),
and optionally the terminal alkyl group R is attached to an aminocarbonyl-aldoxime or a ketoaldoxime to form a bis-functional nucleophile, thereby statistically improving nucleophilic potential;
(4) a compound having the formula: 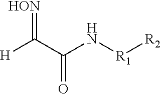
wherein
R₁ is selected from a group consisting of: —(CH₂)ₙ—, and (CH₂)ₙ—CH(CH₃)—, wherein N is the integer 0, 1, 2, 3, 4 or 5;
R₂ is selected from a group consisting of a compound having a structure as set forth in the Summary:
and any combination thereof);
(5) a compound having the formula: 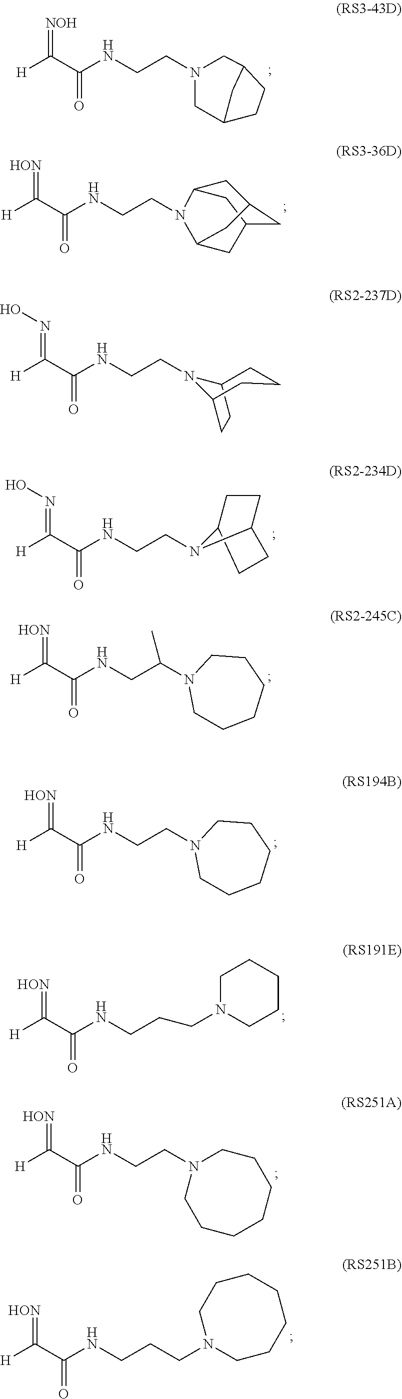 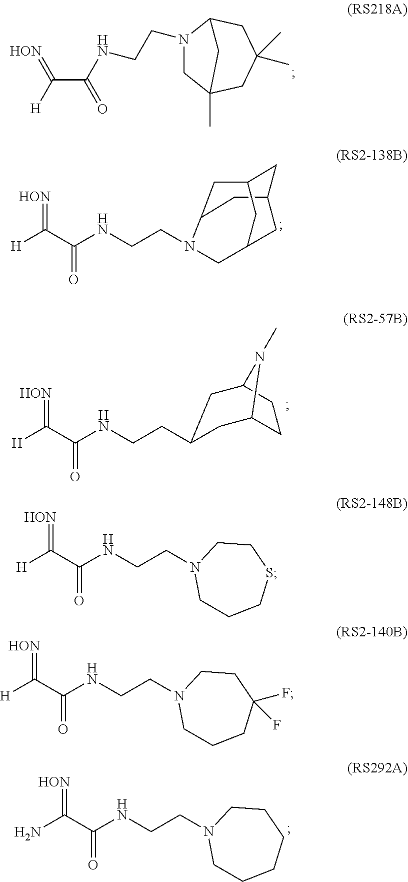
and
a combination thereof;
(6) compound having a structure as set forth in Table 1 (FIG. 3) to Table 2 (FIG. 4), or equivalents thereof, or pharmaceutically acceptable salt thereof,
wherein optionally the compound is RS2 138B, RS194B or RS191E;
wherein optionally the compound is RS251A, RS251B, RS218A, or RS2-57B; or
wherein optionally the compound is RS2-148B, RS2-140B, RS3-43D, RS3-36D, RS2-237D, RS2-234D or RS2-245C (FIG. 4A);
(7) any combination of any of the compounds of (1) to (6); or
(8) an isomer, an optical isomer or a stereoisomer; a racemate or a racemic mixture, an enantiomer, an individual diastereomer or a diastereomeric mixture or an analog, a crystalline product or a crystalline intermediate, a pharmaceutically acceptable salt, or a prodrug of the structure or compound, or a bioisostere of any compound or structure of (1) to (7); and
(b) a brain efflux transporter inhibitor, or an inhibitor of renal tubular secretion.

2: A formulation comprising a compound or composition as set forth in claim 1, wherein optionally the formulation is a solid, liquid, gel, aerosol, powder or emulsion formulation.

3: A pharmaceutical composition comprising a therapeutic combination as set forth in claim 1, wherein optionally the pharmaceutical composition is formulated for enteral or parenteral administration.

4: A pump, a device, a subcutaneous infusion device, a continuous subcutaneous infusion device, an infusion pen, a needles, a reservoir, an ampoules, a vial, a syringe, a cartridge, a disposable pen or jet injector, a prefilled pen or a syringe or a cartridge, a cartridge or a disposable pen or jet injector, a two chambered or multi-chambered pump, a syringe, a cartridge or a pen or a jet injector, comprising a therapeutic combination as set forth in claim 1.

5: A method for treating, ameliorating or protecting (preventing) an organophosphate toxicity or poisoning or toxic exposure, or for treating, ameliorating or protecting (preventing) organophosphate inhibition of an acetylcholinesterase (AChE), comprising:
administering to a patient or an individual in need thereof, a therapeutic combination as set forth in claim 1, wherein optionally the compound or formulation is administered enterally or parenterally,
wherein optionally the compound or formulation is administered orally, sublingually, parenterally, by inhalation spray, nasally, topically, intrathecally, intrathecally, intracerebrally, epidurally, intracranially or rectally, or
administering the therapeutic combination as set forth in claim 1, using a pump, a device, a subcutaneous infusion device, a continuous subcutaneous infusion device, an infusion pen, a needles, a reservoir, an ampoules, a vial, a syringe, a cartridge, a disposable pen or jet injector, a prefilled pen or a syringe or a cartridge, a cartridge or a disposable pen or jet injector, a two chambered or multi-chambered pump, a syringe, a cartridge or a pen or a jet injector.

6: The method of claim 5, wherein the organophosphate toxicity, poisoning or toxic exposure is caused by exposure of the patient or individual to an alkyl methylphosphonate or related nerve agent, or an alkylphosphorate insecticide,
and optionally the organophosphate (OP) is or is a component of a toxin, an herbicide, an insecticide, or a nerve gas or nerve agent,
and optionally the organophosphate (OP) is or comprises parathion, malathion, methyl parathion, chlorpyrifos, diazinon, dichlorvos, phosmet, fenitrothion, tetra-chlorvinphos, azamethiphos or azinphos methyl,
or the nerve agent is or comprises VX (or ethyl ({2-[bis(propan-2-yl)amino]ethyl}sulfanyl)(methyl)phosphinate), paraoxon, soman (O-Pinacolyl methylphosphonofluoridate), tabun (Ethyl N,N Dimethyl-phosphoramido-cyanidate), sarin ((RS)-propan-2-yl methyl-phosphono-fluoridate) or a combination thereof.

7: The method of claim 5, wherein the acetylcholinesterase (AChE) is in the central nerve system (CNS), or the acetylcholinesterase (AChE) is a human acetylcholinesterase (hAChE).

8: A method for treating, preventing or ameliorating excessive acetylcholine stimulation in the brain, comprising:
administering to a patient or an individual in need thereof, a therapeutic combination as set forth in claim 1,
wherein optionally the compound or formulation is administered enterally or parenterally,
wherein optionally the compound or formulation is administered orally, sublingually, parenterally, by inhalation spray, nasally, topically, intrathecally, intrathecally, intracerebrally, epidurally, intracranially or rectally, or
administering the therapeutic combination as set forth in claim 1, using a pump, a device, a subcutaneous infusion device, a continuous subcutaneous infusion device, an infusion pen, a needles, a reservoir, an ampoules, a vial, a syringe, a cartridge, a disposable pen or jet injector, a prefilled pen or a syringe or a cartridge, a cartridge or a disposable pen or jet injector, a two chambered or multi-chambered pump, a syringe, a cartridge or a pen or a jet injector.

9: The method of claim 8, wherein the excessive acetylcholine stimulation in the brain, the CNS or the PNS is caused by a drug, a drug overdose, or a poisoning or a toxic exposure to a drug, and optionally the drug overdose causing the excessive acetylcholine stimulation is caused at least in part by physostigmine, neostigmine, pyridostigmine, diisopropylfluorophosphate and/or echothiophate.

10. (canceled)

11: The method of claim 8, wherein:
(a) the nucleophilic hydroxyimino-acetamido alkylamine antidotes and the brain efflux transporter inhibitor and/or the inhibitor of renal tubular secretion or administered together (or substantially at the same time), and/or are formulated together; or
(b) the nucleophilic hydroxyimino-acetamido alkylamine antidotes and the brain efflux transporter inhibitor and/or the inhibitor of renal tubular secretion or administered separately, and/or are formulated separately.

12. (canceled)

13: The therapeutic combination of claim 1, wherein the brain efflux transporter inhibitor, or an inhibitor of renal tubular secretion, comprises or is an organic anion transporter (OAT) inhibitor and/or an organic cation (OCT) transporter inhibitor.

14: The therapeutic combination of claim 1, wherein the brain efflux transporter inhibitor, or an inhibitor of renal tubular secretion, comprises or is
(i) metformin (or GLUCOPHAGE™); a fluoroquinolone antibiotic (wherein optionally the fluoroquinolone is ciprofloxacin, or CILOXAN™, CIPRO™, or NEOFLOXIN™), norfloxacin (or NOROXIN™), levofloxacin (or LEVAQUIN™), or a combination thereof,
(ii) a p-glycoprotein inhibitor, wherein optionally the p-glycoprotein inhibitor is or comprises: tariquidar, clarithromycin (or BIAXIN™), erythromycin (or ERYTHROCIN™), ritonavir (or NORVIR™), verapamil (or CALAN™, COVERA™ VERELAN™, TARKA™), gallopamil (or methoxyverapamil), dimeditiapramine (or tiapamil), diltiaZem (or CARDIZEM™), amiodarone (or CORDARONE™ NEXTERONE™), ciclosporin (or NEORAL™, SANDIMMUNE™), colchicine (or COLCRYS™, MITIGARE™), felodipine (or PLENDIL™), ketoconazole (or NIZORAL™), lansoprazole (or PREVACID™), omeprazole (or LOSEC™, PRILOSEC™, ZEGERID™), nifedipine (or ADALAT™, PROCARDIA™), paroxetine (or PAXIL™, SEROXAT™), reserpine, saquinavir (or INVIRASE™ FORTOVASE™), sertraline (or ZOLOFT™), quinidine (or QUINAGLUTE™ QUINIDEX™), tamoxifen (or NOLVADEX™, GENOX™, TAMIFEN™), duloxetine (or CYMBALTA, ARICLAIM), loperamide (or IMODIUM™), azidothymidine (or zidovudine, RETROVIR™) or a homodimer thereof, elacridar, zosuquidar, laniquidar, valspodar, reversan, or a combination thereof;
(iii) an antihistamine, wherein optionally the antihistamine is fexofenadine (or ALLEGRA™), loratadine (or CLARITIN™), hydroxyzine (or ATARAX™) diphenhydramine (or BENADRYL™), acrivastine or cetirizine;
(iv) an ATP-binding cassette (ABC) inhibitor, wherein optionally the ABC inhibitor is a tyrosine kinase (TK) inhibitor (TKI), and optionally the TKI comprises imatinib (or GLEEVEC™, GLIVEC™), gefitinib (or IRESSA™), erlotinib (or TARCEVA™), ceritinib (or ZYKADIA™), lenvatinib (or LENVIMA™, LENVANIX™) or a small inhibitory RNA (siRNA) that inhibits an ABC or a TK; or
(v) any combination of (i) to (iv).
